(19) United States
(12) Patent Application Publication
(10) Pub. No.: US 2026/0307062 A1
(43) Pub. Date: Oct. 8, 2026

(54) DIELECTRIC MIE RESONATOR CUPRIC/CUPROUS-OXIDE BASED PHOTOCATALYSTS FOR SUZUKI COUPLING REACTION

(71) Applicant: UNIVERSITY OF SOUTH FLORIDA, Tampa, FL (US)

(72) Inventors: Marimuthu Andiappan, Tampa, FL (US); Dharendra Yogi Goswami, Tampa, FL (US); Giovanna Coelho Moreira Pereira, Tampa, FL (US)

(21) Appl. No.: 19/638,038

(22) Filed: Apr. 2, 2026

Related U.S. Application Data
(60) Provisional application No. 63/782,158, filed on Apr. 2, 2025.

(51) Int. Cl. B01J 23/89; B01J 8/08; B01J 35/30; B01J 35/39; B01J 35/40; B01J 35/45; B01J 35/53; B01J 37/02; B01J 37/04; B01J 37/06; B01J 37/16; C07C 1/32

(52) U.S. Cl. CPC B01J 23/8926; B01J 8/08; B01J 35/39; B01J 35/397; B01J 35/40; B01J 35/45; B01J 35/53; B01J 37/0225; B01J 37/04; B01J 37/06; B01J 37/16; C07C 1/321; B01J 2208/00805

(57) ABSTRACT

Disclosed herein are hybrid nanocatalysts that include a copper-containing light-harvesting core particle and palladium disposed at or near a surface of the core particle. In representative embodiments, the core particle comprises cuprous oxide (Cu₂O), cupric oxide (CuO), metallic copper (Cu⁰), and/or mixtures thereof and exhibits a dielectric Mie resonance feature in a 400-600 nm region. Palladium is present as nanoclusters, islands, and/or a thin film or partial thin film at or near the core surface. Under irradiation, the copper-containing core converts incident radiation into heat that accelerates Suzuki cross-coupling between an aryl electrophile and an organoboron reagent without external heating; the nanocatalyst also promotes coupling without irradiation and without external heating with longer reaction times. Broadband irradiation spanning about 300-2000 nm is delivered at about one-sun intensity, and in certain embodiments the irradiation source exhibits peak radiation within about 300-600 nm at the reaction vessel.

### CROSS-REFERENCE TO RELATED APPLICATIONS

This application claims the benefit of and priority to U.S. provisional application Ser. No. 63/782,158 entitled “DIELECTRIC MIE RESONATOR CUPRIC/CUPROUS-OXIDE BASED PHOTOCATALYSTS FOR SUZUKI COUPLING REACTION”, filed Apr. 2, 2025, the entire disclosure of which is hereby incorporated by reference.

### STATEMENT REGARDING FEDERALLY SPONSORED RESEARCH

This invention was made with government support under 2102238, 2102239, and 2237454 awarded by the National Science Foundation. The government has certain rights in the invention.

### FIELD

This disclosure relates to heterogeneous catalysis, photothermal catalysis, and carbon-carbon (C—C) cross-coupling reactions. More particularly, the disclosure relates to copper oxide-based nanostructures decorated with palladium (Pd) nanoclusters and their use to catalyze Suzuki-type cross-coupling under broadband irradiation and/or without external heating.

### BACKGROUND

Carbon-carbon bond-forming reactions are widely used in the synthesis of pharmaceuticals, agrochemicals, and functional polymers. Suzuki cross-coupling between an organoboron reagent and an aryl or vinyl halide is a prominent Pd-catalyzed route to biaryl structures. In many large-scale settings, Suzuki coupling uses homogeneous Pd catalysts and thermal energy inputs, often at elevated temperatures. Homogeneous Pd systems can present challenges in catalyst recovery and reuse and can contribute to metal contamination concerns.

Photothermal catalysis uses incident electromagnetic radiation (including solar irradiation) to generate heat that drives thermally activated reactions. Copper (I) oxide (Cu₂O) and copper (II) oxide (CuO) are earth-abundant semiconductors with broad optical absorption. When configured as optically resonant nanostructures, copper oxides can harvest broadband light and convert absorbed light into heat. However, copper oxides alone do not provide the Pd redox cycling typically required for high-efficiency Suzuki coupling and therefore benefit from an active Pd component.

### SUMMARY

The disclosure provides hybrid nanocatalysts comprising (i) a copper oxide core material selected from Cu₂O, CuO, metallic copper (Cu⁰) or mixtures thereof and (ii) palladium disposed at or near a surface of the copper oxide core, for example as Pd nanoclusters, nanoparticles, islands, discontinuous coatings, or combinations thereof. The Pd component is present at ultra-low loading relative to the copper oxide support while providing catalytically active sites for cross-coupling.

Under broadband irradiation (e.g., about 300-2000 nm), the copper-containing core absorbs incident light and converts a portion of the absorbed energy into heat. The resulting localized and/or bulk heating accelerates coupling reactions at Pd sites (photothermal catalysis). The disclosed hybrid nanocatalysts also promote coupling without irradiation and without external heating, albeit with slower kinetics.

In illustrative examples, Cu₂O—Pd and CuO—Pd hybrid nanocatalysts catalyze coupling of iodobenzene and phenylboronic acid to form biphenyl at approximately one-sun intensity (~107.5 mW/cm²) using a broadband ELH 120V-300W lamp. Near-quantitative yields are achieved within minutes (e.g., about 10 minutes) and complete yield is achieved within about one hour of irradiation without external heating. Thermal control experiments conducted at matched bulk temperatures indicate that the observed enhancement under irradiation is attributable primarily to photothermal heating rather than a direct photochemical pathway.

### BRIEF DESCRIPTION OF THE FIGURES

FIG. 1 includes representative electron microscopy and compositional mapping of copper oxide-palladium hybrid nanocatalysts. (A) Illustrates a representative scanning electron microscopy (SEM) image of a Cu₂O—Pd hybrid nanocatalyst showing particle morphology and size. (B) Illustrates a representative SEM image of a CuO—Pd hybrid nanocatalyst showing particle morphology and size. (C) Illustrates a representative elemental mapping dataset (e.g., EDS) for Cu₂O—Pd showing spatial distribution of copper, oxygen, and palladium across particles. (D) Illustrates a representative elemental mapping dataset (e.g., EDS) for CuO—Pd showing spatial distribution of copper, oxygen, and palladium across particles. (E) Illustrates a representative high-resolution TEM image showing Pd lattice fringes (e.g., Pd(111)) associated with Pd nanoclusters on a Cu₂O particle. (F) Illustrates a representative high-resolution TEM image showing Pd lattice fringes (e.g., Pd(111) and Pd(200)) associated with Pd nanoclusters on a CuO particle.

FIG. 2 is a characterization of structural and optical properties of selected nanocatalysts. (A) Illustrates an X-ray diffraction (XRD) pattern of a Cu₂O—Pd hybrid nanocatalyst. (B) Illustrates an XRD pattern of a CuO—Pd hybrid nanocatalyst. (C) Illustrates a UV-Vis extinction spectrum of the Cu₂O—Pd system (e.g., catalyst dispersion in reaction mixture), including optical features in the 300-1000 nm region. (D) Illustrates a UV-Vis extinction spectrum of the CuO—Pd system (e.g., catalyst dispersion in reaction mixture), including optical features in the 300-1000 nm region.

FIG. 3 shows finite-difference time-domain (FDTD) simulation. (A) Illustrates a FDTD simulated extinction cross section as a function of incident wavelength for Cu₂O—Pd nanospheres in a first particle-size range, using an effective Pd shell approximation. (B) Illustrates an FDTD simulated extinction cross section as a function of incident wavelength for Cu₂O—Pd nanospheres in a second particle-size range. (C) Illustrates a simulated electric-field intensity enhancement map at a dielectric resonance wavelength for a representative Cu₂O—Pd nanosphere. (D) Illustrates a simulated magnetic-field intensity enhancement map at a dielectric resonance wavelength for the representative Cu₂O—Pd nanosphere.

FIG. 4 shows representative volume normalized absorption. (A) Illustrates an FDTD simulated volume-normalized absorption cross section as a function of wavelength for Cu₂O—Pd nanospheres across a first size grouping. (B) Illustrates an FDTD simulated volume-normalized absorption cross section as a function of wavelength for Cu₂O—Pd nanospheres across a second size grouping. (C) Illustrates an FDTD simulated volume-normalized absorption cross section as a function of wavelength for Cu₂O—Pd nanospheres across a third size grouping. (D) Illustrates a summary plot of relative absorbed power versus particle size (e.g., a volcano plot) derived from broadband integration of the volume-normalized absorption response.

FIG. 5 illustrates catalytic performance data for Suzuki coupling under light-on and light-off (ambient) conditions. Conversion and yield are plotted as a function of time for Cu₂O—Pd and CuO—Pd, including replicate variability/error bars where shown. (A) Iodobenzene conversion in the presence of Cu₂O—Pd. (B) Biphenyl yield in the presence of Cu₂O—Pd. (C) Iodobenzene conversion in the presence of CuO—Pd. (D) Biphenyl yield in the presence of CuO—Pd.

FIG. 6 illustrates catalytic performance data comparing light-on (photothermal) operation to light-off with external thermal input (temperature-matched control). Conversion and yield are plotted as a function of time for Cu₂O—Pd and CuO—Pd under the indicated operating modes. (A) Iodobenzene conversion in the presence of Cu₂O—Pd. (B) Biphenyl yield in the presence of Cu₂O—Pd. (C) Iodobenzene conversion in the presence of CuO—Pd. (D) Biphenyl yield in the presence of CuO—Pd.

FIG. 7 illustrates characterizations of catalyst stability. (A) XRD patterns of Cu₂O—Pd nanocatalysts before and after the reaction. (B) XRD patterns of CuO—Pd nanocatalysts before and after the reaction. (C) TEM images of Cu₂O—Pd nanocatalysts after the reaction. (D) TEM images of CuO—Pd nanocatalysts after the reaction.

FIG. 8 includes a spectral comparison between the broadband lamp source and the solar spectrum, presented as spectral radiance/intensity versus wavelength, demonstrating overlap across relevant wavelength ranges.

FIG. 9 shows representative TEM images. (A) TEM image of hybrid Cu₂O—Pd nanocatalysts. (B) TEM image of hybrid CuO—Pd nanocatalysts.

FIG. 10 shows high-angle annular dark-field stem (HAADF-STEM) microscopy images. (A) HAADF-STEM image of Cu₂O—Pd nanocatalyst. (B-E) Respective EDS individual elemental mapping. (e.g., Cu, O, Pd), illustrating spatial distribution of palladium relative to copper oxide for a first hybrid catalyst sample.

FIG. 11 includes representative HAADF and/or related microscopy images with corresponding elemental maps (e.g., Cu, O, Pd), illustrating spatial distribution of palladium relative to copper oxide for a second hybrid catalyst sample. (A) HAADF-STEM image of CuO—Pd nanocatalyst and (B-E) respective EDS individual elemental mapping.

FIG. 12 illustrates FDTD-simulated extinction cross section as a function of incident light wavelength of nanoparticles each incorporating a 1 nm Pd shell. (A) Extinction spectra of CuO—Pd nanospheres ranging from 40 to 180 nm in diameter (20 nm intervals). (B) Extinction spectra of CuO—Pd nanospheres ranging from 180 to 300 nm in diameter (20 nm intervals.

FIG. 13 includes additional simulated extinction spectra (two size regimes shown) for Pd-decorated copper oxide nanospheres as a function of wavelength, providing comparative size-dependent optical response. (A) Nanoparticle size of 40-180 nm. (B) Nanoparticle size of 180-300 nm.

FIG. 14 includes simulated extinction spectra for copper oxide nanospheres (without Pd decoration) across multiple sizes as a function of wavelength, enabling comparison against Pd-decorated hybrids. (A) Nanoparticle size of 40-180 nm. (B) Nanoparticle size of 180-300 nm.

FIG. 15 includes simulated absorption cross-section spectra as a function of wavelength for multiple particle sizes (panels corresponding to different materials and/or Pd-decorated vs. non-decorated configurations), supporting analysis of broadband photothermal absorption behavior. (A) Shows a simulated absorption cross-section (or related absorption metric) as a function of incident light wavelength for a first copper oxide-Pd nanoparticle configuration across a range of particle sizes. (B) Shows a simulated absorption cross-section (or related absorption metric) as a function of incident light wavelength for a second copper oxide-Pd nanoparticle configuration across a range of particle sizes. (C) Shows a simulated absorption cross-section (or related absorption metric) as a function of incident light wavelength for a third copper oxide-Pd nanoparticle configuration across a range of particle sizes. Together, subpanels (A)-(C) provide comparative absorption behavior relevant to photothermal light harvesting under broadband irradiation.

FIG. 16 includes additional simulated absorption cross-section spectra as a function of wavelength for multiple particle sizes (panels corresponding to different materials and/or Pd-decorated vs. non-decorated configurations), supporting comparative photothermal absorption analysis. (A) Illustrates a simulated absorption cross-section (or related absorption metric) as a function of incident light wavelength for a first copper oxide-Pd nanoparticle configuration across a range of particle sizes in a second simulation set. (B) Illustrates a simulated absorption cross-section (or related absorption metric) as a function of incident light wavelength for a second copper oxide-Pd nanoparticle configuration across a range of particle sizes in the second simulation set. (C) Illustrates a simulated absorption cross-section (or related absorption metric) as a function of incident light wavelength for a third copper oxide-Pd nanoparticle configuration across a range of particle sizes in the second simulation set. Together, subpanels (A)-(C) provide additional comparative absorption behavior supporting analysis of photothermal conversion efficiency across wavelength and particle size.

FIG. 17 includes simulated volume-normalized absorption spectra across particle sizes and a summary plot of relative absorbed power versus particle size for Pd-decorated copper oxide, identifying an optimal size window for broadband absorption. (A) Illustrates a simulated volume-normalized absorption cross-section (or related normalized absorption metric) as a function of incident light wavelength for a first grouping of particle sizes. (B) Illustrates a simulated volume-normalized absorption cross-section (or related normalized absorption metric) as a function of incident light wavelength for a second grouping of particle sizes. (C) Illustrates a simulated volume-normalized absorption cross-section (or related normalized absorption metric) as a function of incident light wavelength for a third grouping of particle sizes. Together, subpanels (A)-(C) provide a size-dependent, normalized absorption comparison used to evaluate relative absorbed power under broadband illumination conditions.

FIG. 18 includes temperature-versus-time data for a Cu₂O—Pd-catalyzed reaction mixture under light-on and a comparator light-off condition (as labeled), illustrating heating profiles during reaction operation.

FIG. 19 includes temperature-versus-time data for a CuO—Pd-catalyzed reaction mixture under light-on and a comparator light-off condition (as labeled), illustrating heating profiles during reaction operation.

FIG. 20 includes temperature-versus-time comparisons under light-on conditions for Pd-decorated Cu₂O versus the corresponding copper oxide without Pd, illustrating that copper oxide in this form provides photothermal heating while Pd provides catalytic activity.

FIG. 21 includes temperature-versus-time comparisons under light-on conditions for Pd-decorated CuO versus the corresponding copper oxide without Pd, illustrating that copper oxide provides photothermal heating while Pd provides catalytic activity.

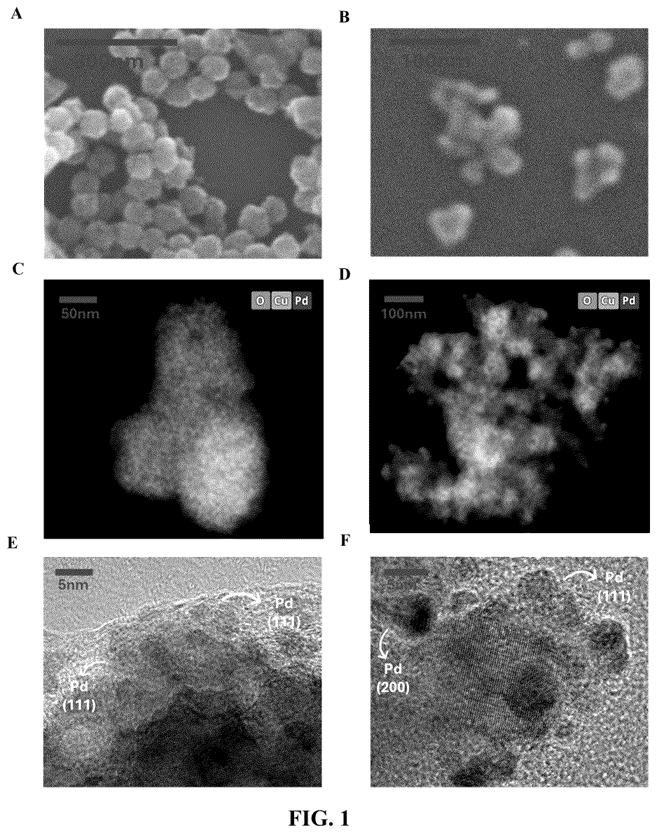

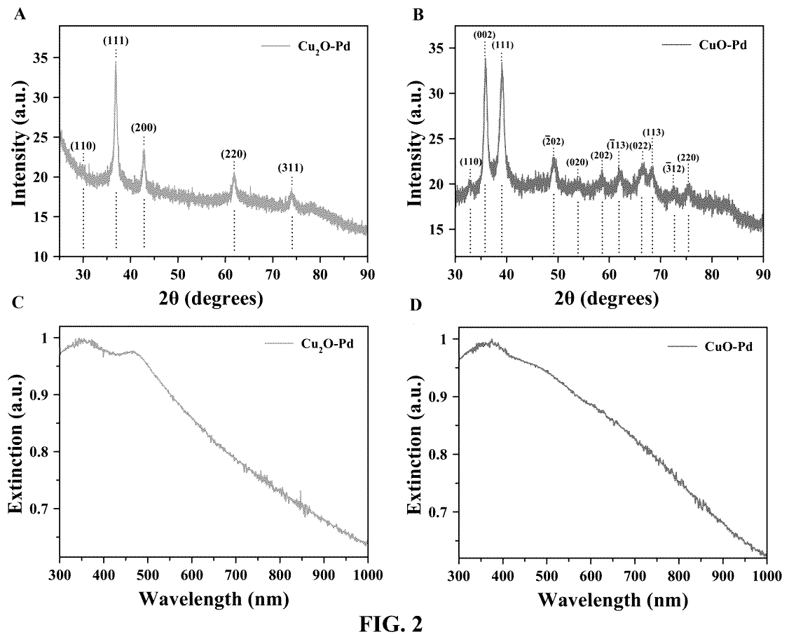

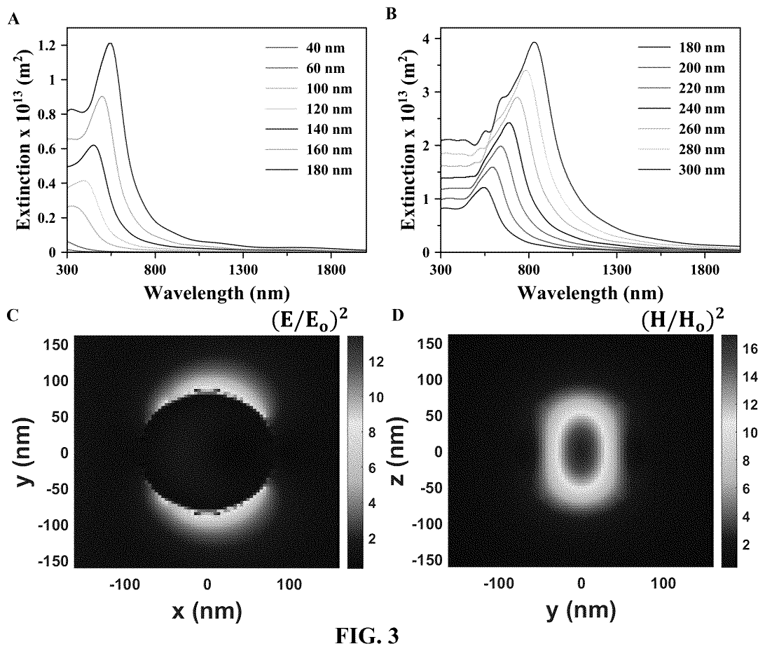

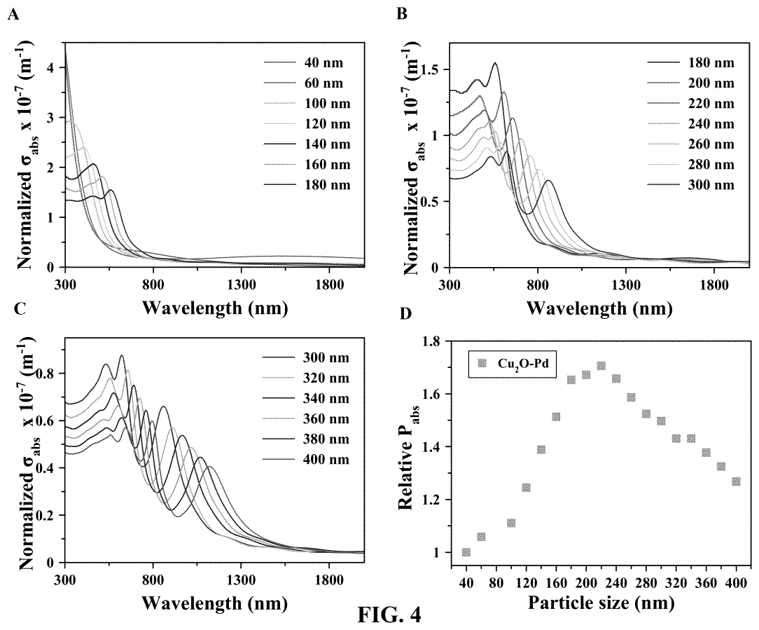

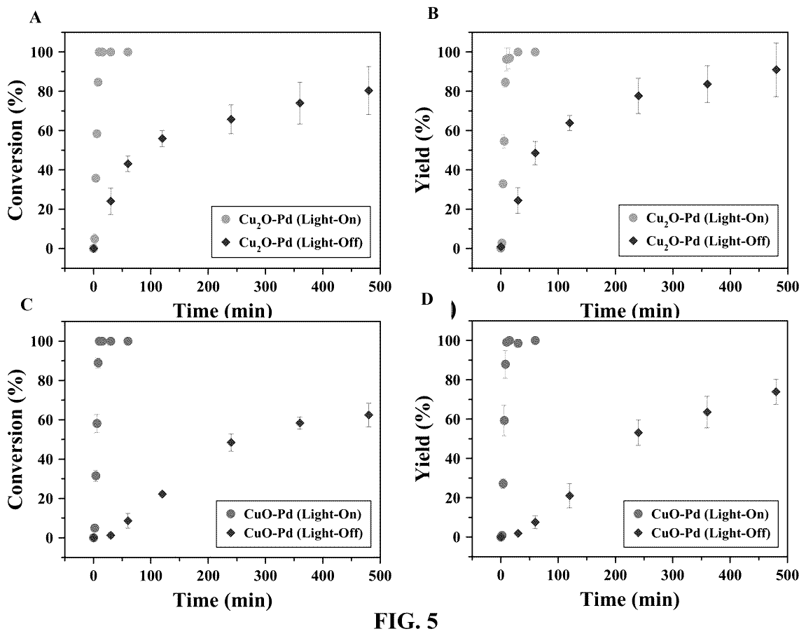

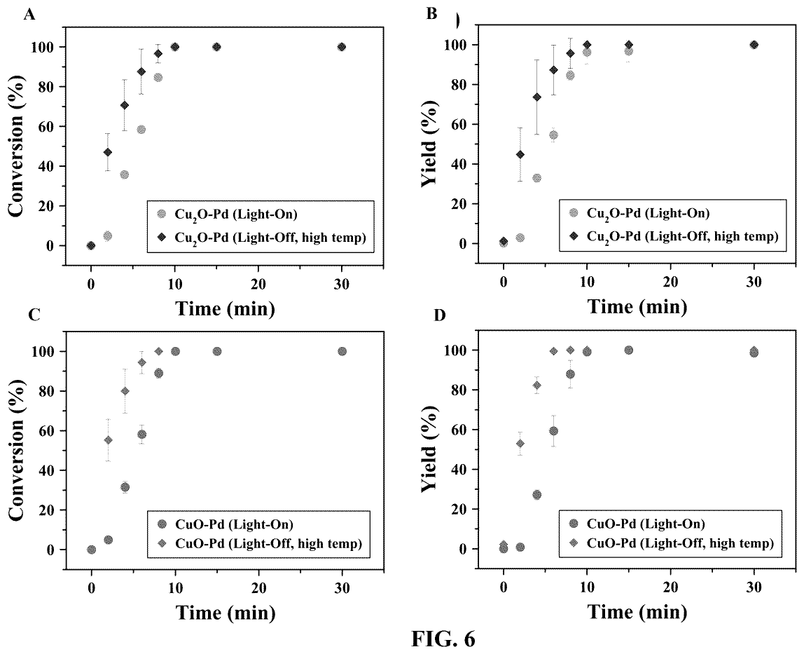

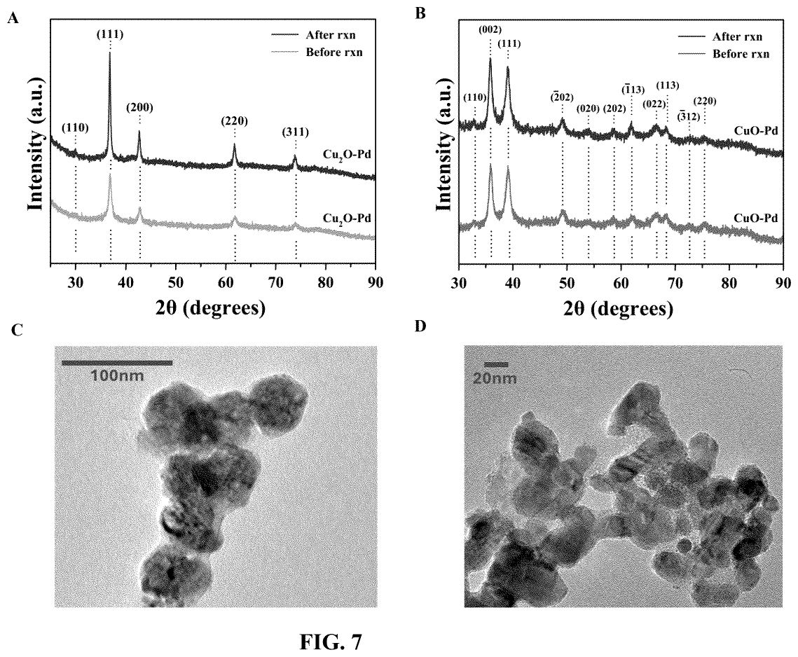

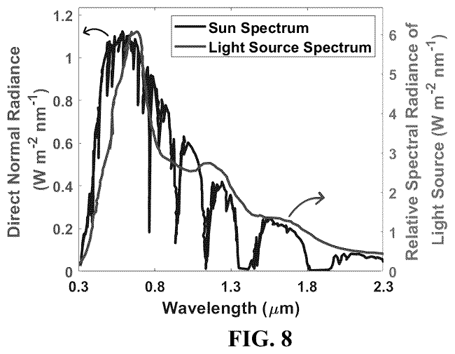

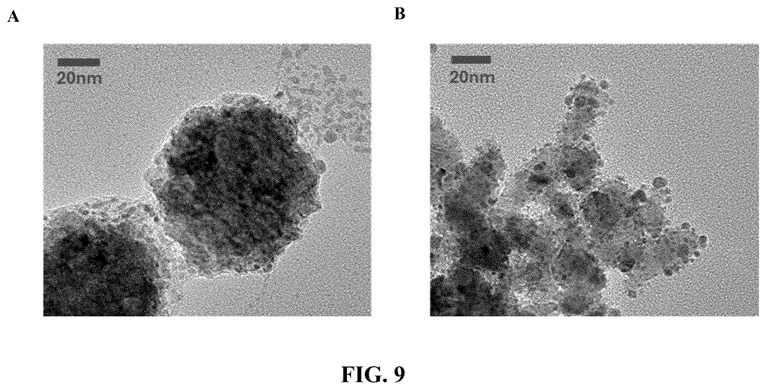

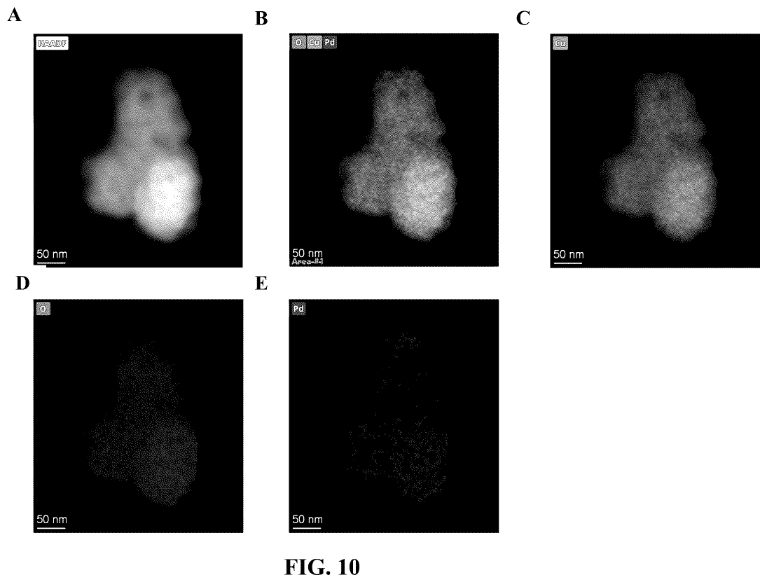

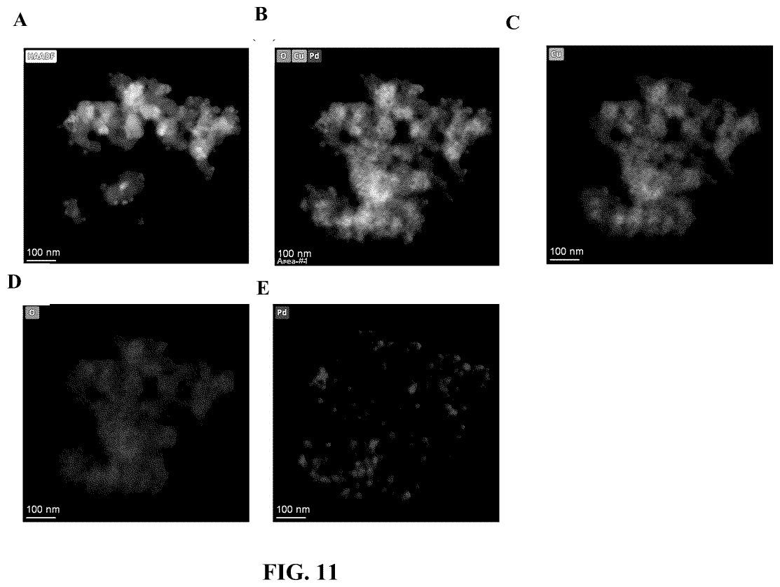

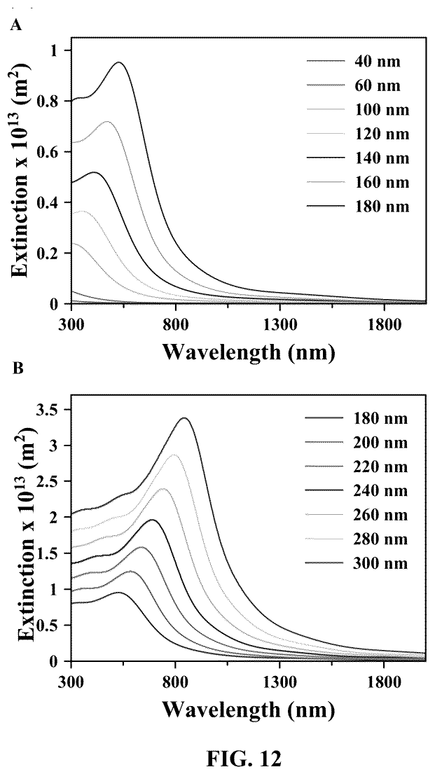

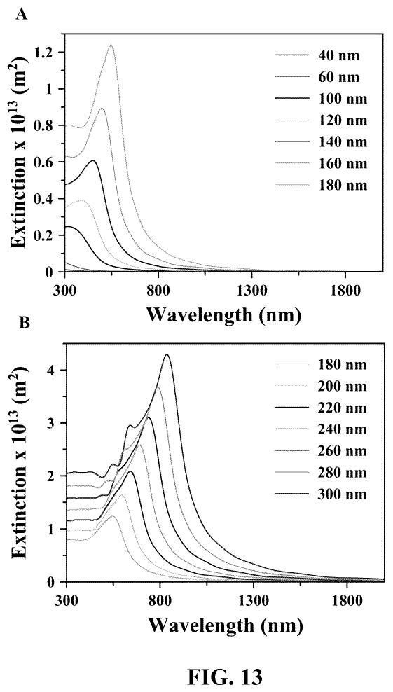

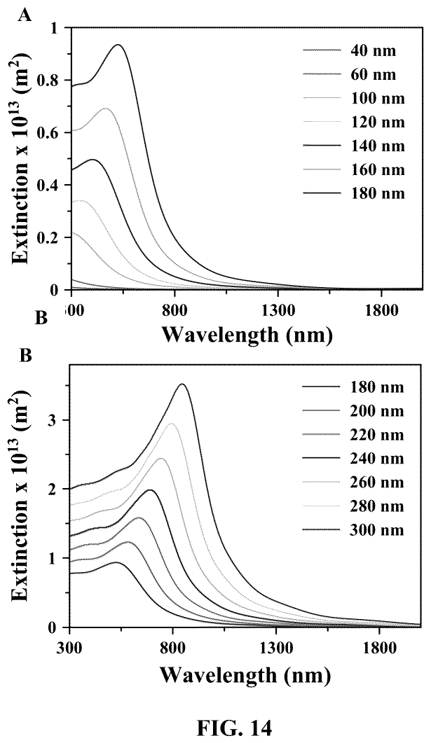

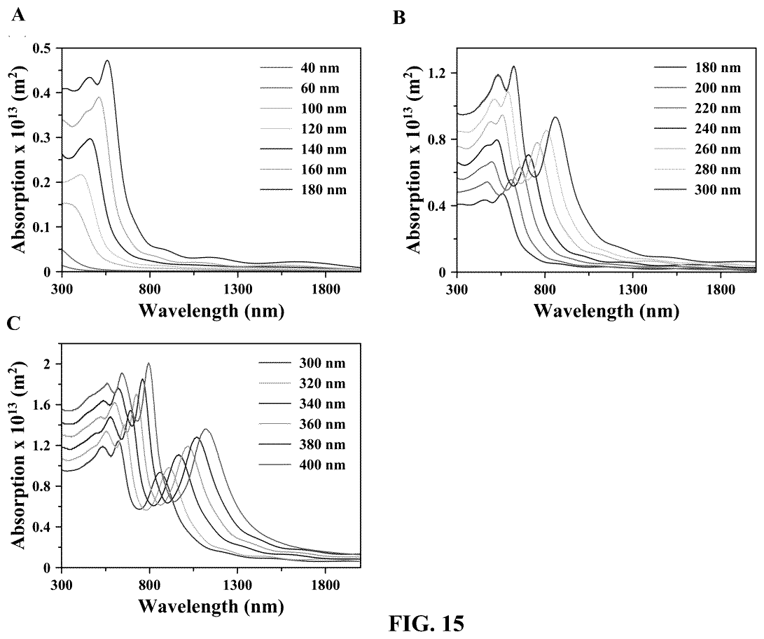

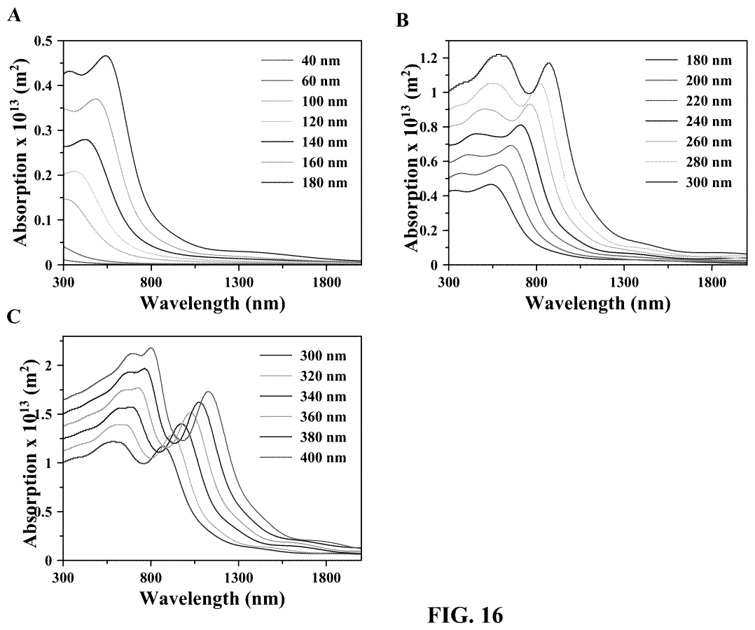

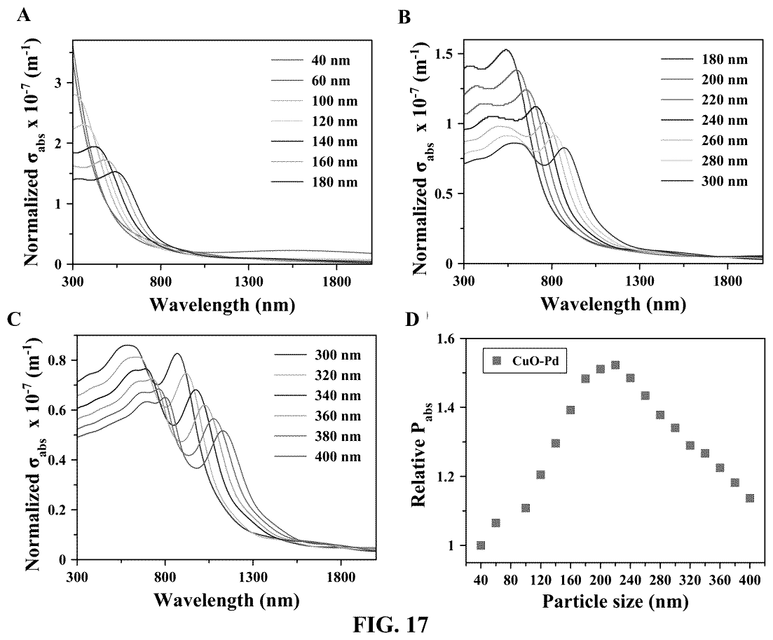

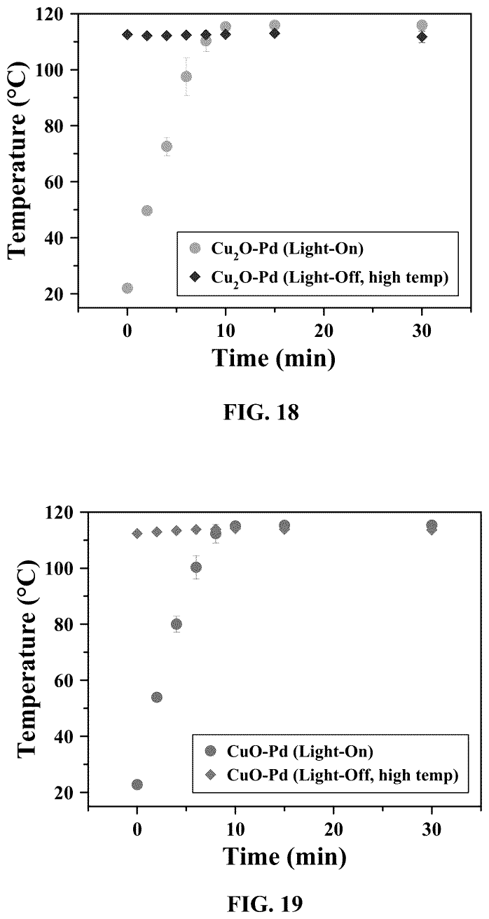

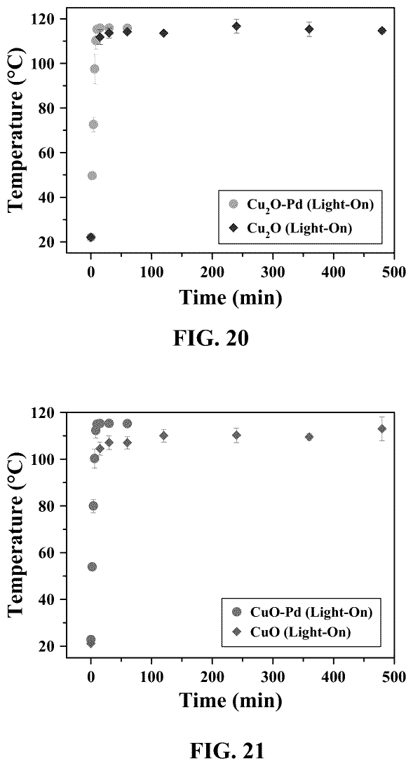

### DEFINITIONS

The following terms, when used in this specification and the appended claims, refer to the meanings set forth below. Where a term has a plain and ordinary meaning to a person having ordinary skill in the art, that meaning applies unless the term has been expressly defined herein.

As used herein, the words “comprising”, “comprises” and “comprised of” refer to open-ended transitional phrases that include the listed elements or steps and further include additional, unrecited elements or steps.

As used herein, the words “including”, “includes” and “include” refer to non-limiting terms that list exemplary features or steps.

As used herein, the word “about” refers to a variation that accounts for typical measurement uncertainty and experimental error for the referenced parameter and measurement method. For example, “about” encompasses variations commonly accepted in the relevant art for instrument precision, sample preparation, and environmental conditions.

As used herein, the word “substantially” refers to a degree of approximation that achieves the stated function or result within practical tolerances.

As used herein, the word “nanoparticle” refers to a particle having at least one characteristic dimension in a range from 1 nm to 1000 nm.

As used herein, the word “nanocatalyst” refers to a catalyst composition that includes nanoparticles and that promotes a chemical transformation via catalytic turnover.

As used herein, the term “hybrid nanocatalyst” refers to a composite catalyst that includes at least a copper oxide component and a palladium component that remain physically associated such that palladium resides at or near a surface of copper oxide particles.

As used herein, “copper oxide” refers to Cu₂O, CuO, mixed-valence copper oxides, copper oxides that include dopants, and combinations thereof.

As used herein, the phrase “palladium disposed at or near a surface” refers to palladium present as nanoclusters, nanoparticles, islands, discontinuous coatings, or combinations thereof on a copper oxide particle surface and/or within a near-surface region accessible to reactants.

As used herein, the phrase “ultra-low palladium loading” refers to palladium present at a loading lower than typical homogeneous Suzuki catalyst loadings for comparable throughput, and includes palladium loadings at or below 1 wt % relative to total catalyst mass, and further includes loadings at or below 0.5 wt % or 0.1 wt % relative to total catalyst mass.

As used herein, “photothermal” refers to conversion of incident electromagnetic radiation into heat, including localized heating at a catalyst surface and/or bulk heating of a reaction mixture.

As used herein, “broadband irradiation” refers to irradiation that includes wavelengths across a range that spans at least visible wavelengths and further includes ultraviolet and/or infrared wavelengths. In one embodiment, broadband irradiation spans 300 nm to 2000 nm.

As used herein, “one-sun intensity” refers to an irradiance comparable to terrestrial solar irradiance and includes an intensity of approximately 100 mW/cm² at the reaction vessel.

As used herein, “light-on condition” refers to a reaction condition conducted under irradiation from a light source.

As used herein, “light-off condition” refers to a reaction condition conducted without irradiation from a light source.

As used herein, “external heating” refers to heat supplied to a reaction mixture from a non-irradiation heat source, including a hot plate, heating mantle, oil bath, block heater, or circulating thermal fluid.

As used herein, “coupling reaction” refers to a reaction that forms a carbon-carbon bond between two coupling partners.

As used herein, “Suzuki coupling” or “Suzuki-Miyaura coupling” refers to a cross-coupling between an organoboron reagent and an aryl or vinyl electrophile, typically in the presence of a base and a palladium catalyst, to form a carbon-carbon bond.

As used herein, “aryl halide” refers to an aryl electrophile that includes a halogen substituent (F, Cl, Br, or I) and further includes aryl pseudohalides such as triflates and related leaving groups.

As used herein, “organoboron reagent” refers to a boron-containing coupling partner that includes boronic acids, boronate esters, trifluoroborates, and related organoboron derivatives.

As used herein, “base” refers to a basic reagent that promotes transmetalation and/or related steps in a coupling cycle and includes carbonates, phosphates, hydroxides, alkoxides, and amines.

As used herein, “solvent system” refers to one solvent or a mixture of solvents used to dissolve or suspend reactants and catalyst; the solvent system includes polar aprotic solvents, water, and mixtures thereof.

As used herein, “Mie resonance” or “dielectric Mie resonance” refers to an optical resonance supported by dielectric or semiconducting particles that enhances electromagnetic field intensity within and/or near the particle at specific wavelengths and that increases light-matter interaction relative to non-resonant particles of different size or shape.

As used herein, “conversion” refers to consumption of a specified reactant relative to an initial amount and, when expressed as a percentage, equals ([R]ₒ−[R]ₜ)/[R]ₒ×100, where [R]ₒ refers to initial reactant concentration and [R]ₜ refers to reactant concentration at time t.

As used herein, “yield” refers to formation of a specified product relative to a theoretical maximum based on a limiting reactant and, when expressed as a percentage, equals [P]ₜ/[R]ₒ×100, where [P]ₜ refers to product concentration at time t and [R]ₒ refers to an initial concentration of the limiting reactant.

As used herein, “hybrid” refers to a composite that includes at least a copper oxide component and a palladium component that are physically associated such that Pd is disposed at or near a surface of copper oxide particles.

As used herein, “thin film” refers to a layer of palladium disposed on the surface, and/or within a near-surface region, of a copper oxide particle as a continuous coating or a discontinuous coating (including islands), in which the palladium layer has a thickness on the nanometer scale.

As used herein, the term “metallic copper” refers to copper in a zero-valent state (Cu⁰) present as a metal phase, including nanodomains, grains, inclusions, or a surface layer that is distinct from copper oxide phases.

As used herein, the term “copper-containing core” refers to a particle that includes one or more copper oxide phases (Cu₂O and/or CuO) and, in some embodiments, further includes metallic copper (Cu⁰).

As used herein, the phrase “mixture of Cu₂O, CuO, and copper” refers to a composite particle that includes (i) a Cu₂O phase, (ii) a CuO phase, and (iii) a metallic copper phase (Cu⁰), where the phases are present as co-existing domains within a single particle and/or within an agglomerate of primary particles.

As used herein, the term “copper oxide particle” includes a copper-containing particle that comprises at least one copper oxide phase and that optionally further comprises metallic copper (Cu⁰) as part of the same particle or as a near-surface region on the particle.

As used herein, the term “cubical” refers to a particle morphology that is cube-like, including particles having six faces with generally planar facets and edges and corners that approximate a cube. A cubical particle includes particles having truncated corners and/or rounded edges arising from synthesis or post-processing. In one embodiment, a cubical particle has an aspect ratio (largest dimension divided by smallest dimension) of 1.0 to 1.3.

As used herein, the term “cubic” is used interchangeably with “cubical.”

As used herein, the term “peak radiation” with respect to an irradiation source refers to a wavelength band that includes a maximum of the spectral irradiance or spectral radiance of the source at the location of the reaction vessel, as measured at the vessel using a spectroradiometer or equivalent instrument. A ‘peak radiation at 300-600 nm’ means that a maximum of the measured spectral power distribution occurs within the 300-600 nm band.

As used herein, the phrase “spectral power distribution” refers to intensity as a function of wavelength for a light source, optionally expressed as spectral irradiance (W·m-2·nm-1) or spectral radiance in a defined geometry.

As used herein, the phrase “band-limited irradiation” refers to irradiation in which wavelengths outside a selected wavelength band are attenuated by an optical filter, dichroic optic, or source selection.

### DETAILED DESCRIPTION OF THE EMBODIMENTS

The following description is provided to enable practice of the disclosed subject matter. Variations and modifications will be apparent to those skilled in the art and is made without departing from the scope of the claims. Features described in connection with one embodiment may be combined with features of another embodiment unless clearly incompatible.

Unless otherwise indicated, ranges are inclusive of endpoints. The term “about” encompasses typical experimental error and variations that do not materially affect function. When a parameter is described as occurring in a range, each value within the range is contemplated as if expressly described.

In one embodiment, disclosed herein is a hybrid nanocatalyst comprises a copper oxide core material selected from Cu₂O, CuO, Cu₂O/CuO mixtures, doped copper oxides, or combinations thereof, and palladium disposed at or near a surface of the core. The palladium is present as nanoclusters or nanoparticles, or a full or a partial thin film, dispersed on the copper oxide surface; in certain laboratory characterizations, Pd nanoclusters have an average diameter of about 3±1 nm. Copper oxide cores are quasi-spherical; in illustrative preparations, Cu₂O—Pd particles exhibit average diameters of about 71±8 nm and CuO—Pd particles exhibit average diameters of about 23±4 nm.

In one embodiment, the palladium loading is reduced relative to conventional homogeneous catalysts while maintaining activity. For example, Pd is deposited from an aqueous K₂PdCl₄ precursor onto copper oxide particles; in an illustrative preparation, a 21.3 mL aliquot of 0.5 mM K₂PdCl₄ provides approximately 1.13 mg Pd associated with about 32 mg of copper oxide. Due to low Pd content, Pd diffraction peaks are below the detection limit of XRD, while Pd is confirmed by microscopy, EDS mapping, and lattice fringe analysis (e.g., d-spacings consistent with Pd(111) and Pd(200) planes).

In one embodiment, the hybrid nanocatalyst exhibits dielectric Mie resonance behavior, producing optical features in a 400-600 nm region and enhancing interaction with incident light.

In certain embodiments, without relying on a continuous Pd shell, the Pd distribution is modeled as an effective thin Pd layer (e.g., ~1 nm) for purposes of electromagnetic simulations.

In certain embodiments, under specific reaction conditions, primary particles form clusters, shifting effective optical response toward that of larger resonant structures (e.g., an effective response corresponding to ~140-180 nm diameters) while maintaining high surface area from the smaller primary particles.

In certain embodiments, broadband irradiation causes the reaction mixture to warm from ambient temperature to elevated temperature (e.g., about 113-116° C.) during a light-on photothermal run.

In certain embodiments, comparable temperature profiles are observed for copper oxide cores with and without Pd decoration, showing that copper oxide provides much of the light-harvesting and heat generation, while Pd provides coupling activity.

In one embodiment, a method is disclosed comprising contacting an aryl or vinyl halide with an organoboron reagent in the presence of a base, a solvent system, and a hybrid copper oxide-Pd nanocatalyst, and maintaining conditions effective to form a C—C coupled product.

In one embodiment, the method is conducted under photothermal conditions (irradiation without external heating), thermal conditions (external heating in the absence of irradiation), or combined irradiation and external heating.

In one embodiment, the method is conducted without irradiation and without external heating at ambient temperature with extended reaction time.

In one embodiment, aryl/vinyl halides include halides selected from the group consisting of aryl chlorides, bromides, iodides, triflates, pseudohalides and combinations thereof.

In one embodiment, substrates are selected from the group consisting of iodobenzene, bromobenzene, chlorobenzene, 4-bromotoluene, 4-bromoanisole, 4-bromobenzaldehyde, 4-bromobenzonitrile, 4-bromopyridine, 1-bromonaphthalene and combinations thereof.

In one embodiment, organoboron reagents are selected from the group consisting of phenylboronic acid and substituted arylboronic acids (e.g., 4-methylphenylboronic acid, 4-methoxyphenylboronic acid, 4-fluorophenylboronic acid), boronate esters, organoboron reagents and combinations thereof.

In one embodiment, bases are selected from the group consisting of carbonates (e.g., K₂CO₃), phosphates, alkoxides, hydroxides, and amines. Solvents include polar aprotic solvents (e.g., N,N-dimethylformamide (DMF), dimethylacetamide, N-methyl-2-pyrrolidone), alcohols, water, and combinations thereof.

In certain embodiments, the hybrid nanocatalysts are also used for other C—C coupling reactions including Sonogashira coupling, Negishi coupling, Kumada coupling, Stille coupling, and Ullmann-type coupling.

In certain embodiments, the hybrid nanocatalysts are heterogeneous and are separated from reaction mixtures by centrifugation, filtration, decantation, or combinations thereof.

In certain embodiments, recovered catalysts are washed and reused. Stability is assessed by comparing pre- and post-reaction XRD patterns and microscopy images showing no significant phase or morphology changes.

In one embodiment, a hybrid nanocatalyst includes a copper oxide particle that functions as a light-harvesting support and a palladium component disposed at or near a surface of the copper oxide particle.

In one embodiment, the palladium component provides catalytically active sites for carbon-carbon bond formation, and the copper oxide particle provides broadband optical absorption that supports photothermal heating under irradiation.

In one embodiment, the palladium is present on an exterior surface and/or within a near-surface region that remains accessible to reactants during coupling reactions.

In one embodiment, the copper oxide particle includes cuprous oxide (Cu₂O). Cu₂O particles present a semiconducting, optically active platform that absorbs incident light and converts absorbed energy into heat in a reaction medium, while palladium disposed at or near the Cu₂O surface promotes cross-coupling turnover.

In one embodiment, the hybrid structure maintains physical association between Cu₂O and palladium during reaction operation to enable heterogeneous catalyst separation and reuse.

In one embodiment, the copper oxide particle includes cupric oxide (CuO). CuO provides broadband light absorption and photothermal heat generation under irradiation, and palladium disposed at or near the CuO surface provides catalytic sites for coupling reactions.

In one embodiment, the hybrid structure retains CuO phase identity during reaction operation and supports post-reaction recovery of the catalyst from the reaction mixture.

In one embodiment, the palladium component is present as nanoclusters disposed at or near the copper oxide surface.

In one embodiment, the nanoclusters provide high surface area palladium sites while maintaining discontinuous surface coverage on the copper oxide support.

In one embodiment, the palladium nanoclusters exhibit an average size of about 1-10 nm, and the nanoclusters remain distributed across the copper oxide surface to provide multiple accessible catalytic sites.

In one embodiment, the palladium component is present at ultra-low loading relative to total catalyst mass while maintaining catalytic activity.

In one embodiment, palladium loading is less than about 1 wt % relative to total catalyst mass, and the copper oxide constitutes a majority fraction of the catalyst weight. This configuration reduces palladium consumption while retaining heterogeneous handling advantages, including separation of the solid catalyst from the reaction medium after product formation.

In one embodiment, the copper oxide particle has an average diameter from about 5 nm to about 500 nm. Within this range, the particle size supports both optical absorption and practical catalyst dispersion in a reaction medium.

In one embodiment, the copper oxide particles are quasi-spherical and exist as individual particles and/or as clusters that form under reaction conditions while preserving accessible surface area for palladium sites.

In one embodiment, the hybrid nanocatalyst exhibits a dielectric Mie resonance feature in a wavelength region from about 400 nm to about 600 nm.

In one embodiment, the resonance increases light-matter interaction relative to non-resonant particles and enhances photothermal conversion efficiency under irradiation that overlaps the resonance region.

In one embodiment, clustering of primary particles during reaction operation shifts the effective optical response toward that of larger resonant structures while preserving the high surface area associated with smaller primary particles.

In one embodiment, a method of forming a biaryl compound includes contacting an aryl halide and an arylboronic acid with the hybrid nanocatalyst in the presence of a base and a solvent system and irradiating the mixture with broadband light to promote photothermal coupling.

In one embodiment, during irradiation, the copper oxide support absorbs broadband light and converts absorbed energy into heat, increasing reaction temperature and accelerating coupling at palladium sites.

In one embodiment, the method yields a coupled biaryl product while maintaining the catalyst as a separable heterogeneous solid.

In one embodiment, the broadband irradiation includes wavelengths from about 300 nm to about 2000 nm.

In one embodiment, this wavelength range overlaps ultraviolet, visible, and near-infrared regions and supplies incident energy that the copper oxide support converts into heat.

In one embodiment, the irradiation spectrum is selected to overlap copper oxide absorption and, in one embodiment, to overlap a dielectric resonance feature of the hybrid nanocatalyst.

In one embodiment, the method is conducted with broadband irradiation delivered at a light intensity at the reaction vessel from about 50 mW/cm² to about 200 mW/cm².

In one embodiment, this intensity range supports rapid photothermal temperature rise of the reaction mixture while preserving practical laboratory or scale-up illumination geometries.

In one embodiment, the light intensity is measured at the reaction vessel surface and is selected to provide a target bulk temperature profile during the coupling reaction.

In one embodiment, the aryl halide includes iodobenzene and the arylboronic acid includes phenylboronic acid such that the biaryl product includes biphenyl.

In one embodiment, the base includes a carbonate base and the solvent system includes a polar aprotic solvent mixed with water, providing a medium that disperses the heterogeneous catalyst and supports the coupling.

In one embodiment, under photothermal irradiation conditions, the reaction achieves high conversion and high yield on a short time scale relative to ambient, light-off operation.

In one embodiment, the method is conducted without external heating.

In one embodiment, the reaction mixture is heated by photothermal conversion arising from broadband irradiation absorbed by the copper oxide support, and the coupling proceeds under this irradiation-driven temperature profile.

In one embodiment, eliminating external heating reduces reliance on fossil-fuel-derived process heat while retaining rapid coupling kinetics under irradiation.

In one embodiment, the reaction mixture reaches a bulk temperature of at least about 80° C. under irradiation.

In one embodiment, the bulk temperature rise results from photothermal conversion by the copper oxide support and correlates with accelerated coupling kinetics at palladium sites.

In one embodiment, temperature is monitored in the reaction mixture and is maintained within a target operating window by controlling light intensity, irradiation distance, stirring, and reactor heat transfer.

In one embodiment, the disclosed hybrid nanocatalyst is used to catalyze a coupling reaction selected from Sonogashira, Negishi, Kumada, Stille, or Ullmann-type coupling.

In one embodiment, the method includes contacting appropriate coupling partners with the hybrid nanocatalyst and irradiating with broadband light to generate photothermal heating that increases reaction temperature at the catalyst interface.

In one embodiment, the copper oxide support provides the photothermal heat source under irradiation and palladium provides catalytic sites that promote the selected coupling reaction under the resulting temperature profile.

In one embodiment, the copper-containing core included co-existing copper phases comprising Cu₂O, CuO, and metallic copper (Cu⁰). The phases were present as discrete nanodomains within a single particle and/or as a composite formed by intimate association of primary particles.

In one embodiment, the copper-containing core included a Cu₂O/CuO matrix and metallic copper nanodomains dispersed within the matrix. The metallic copper nanodomains were located in an interior region of the particle, at grain boundaries, and/or at a near-surface region.

In one embodiment, the copper-containing core included a metallic copper region and a copper oxide region, and the metallic copper region and copper oxide region formed a heterojunction that supported charge redistribution under irradiation.

In one embodiment, the copper-containing core included a metallic copper near-surface region that was covered by an oxide-rich outer region, where the oxide-rich outer region included Cu₂O, CuO, or a mixture of Cu₂O and CuO.

In one embodiment, the copper-containing core included a mixture of Cu₂O, CuO, and metallic copper (Cu⁰), and palladium was disposed at or near a surface of the copper-containing core as nanoclusters, islands, or a discontinuous film.

In one embodiment, metallic copper formed by partial reduction of a copper oxide phase during catalyst preparation and/or during subsequent processing, producing a composite that included Cu₂O, CuO, and metallic copper (Cu⁰).

In one embodiment, metallic copper formed during reaction operation under conditions that established a local reducing environment at the copper-containing core surface, including conditions created by photothermal heating under broadband irradiation in the presence of organic reactants and/or base.

In one embodiment, metallic copper formed in situ by redox cycling of copper at the particle surface during repeated use and recovery, yielding a stable mixture of Cu₂O, CuO, and metallic copper domains while palladium remained disposed at or near the surface.

In one embodiment, the metallic copper phase contributed to heat generation and/or electron transport within the copper-containing core under broadband irradiation, while palladium provided catalytically active sites for carbon-carbon coupling.

In one embodiment, the mixture of Cu₂O, CuO, and metallic copper stabilized optical response of the copper-containing core during reaction operation, and the copper-containing core retained structural integrity during catalyst recovery and reuse.

In one embodiment, the copper-containing core comprised a mixture of Cu₂O, CuO, and metallic copper (Cu⁰).

In one embodiment, the copper-containing core comprised metallic copper and CuO.

In one embodiment, the copper-containing core comprised metallic copper and Cu₂O.

In one embodiment, the metallic copper constituted 0.1 wt % to 50 wt % of the copper-containing core, and the remainder comprised Cu₂O, CuO, or a mixture of Cu₂O and CuO. In one embodiment, the copper-containing core particle is cubical.

In one embodiment, the copper-containing core particle comprises Cu₂O and is cubical. In one embodiment, the copper-containing core particle comprises CuO and is cubical. In one embodiment, the copper-containing core particle comprises a mixture of Cu₂O and CuO and is cubical.

In one embodiment, the copper-containing core particle comprises a mixture of Cu₂O, CuO, and metallic copper (Cu₀) and has a cubical morphology.

In one embodiment, the cubical copper-containing core particle has an average cube edge length of about 10 nm to about 1000 nm; in certain embodiments about 20 nm to about 500 nm; and in certain embodiments about 40 nm to about 300 nm.

In one embodiment, the hybrid nanocatalyst includes a mixture of particle morphologies comprising quasispherical particles and cubical particles, where palladium remains disposed at or near surfaces of both morphologies.

In one embodiment, cubical copper-containing core particles are formed using a shape-directing synthesis that promotes faceted growth, including (i) a solution-phase synthesis that employs a capping agent and/or halide ions to control facet stabilization, (ii) a polyol synthesis, (iii) a hydrothermal or solvothermal synthesis, and/or (iv) a template-assisted synthesis; and subsequently depositing palladium from a palladium (II) precursor to provide the hybrid nanocatalyst.

In one embodiment, cubical Cu₂O particles are converted, at least in part, to CuO by controlled oxidation while retaining cubical morphology, thereby providing a cubical copper-containing core comprising Cu₂O, CuO, or mixtures thereof.

In one embodiment, cubical copper-containing core particles form clusters during reaction operation while maintaining an effective optical response attributable to dielectric Mie resonance, and palladium sites remain accessible at or near particle surfaces.

In one embodiment, the reaction mixture is irradiated with broadband irradiation that includes wavelengths from about 300 nm to about 2000 nm, and the irradiation source exhibits peak radiation in a wavelength band from about 300 nm to about 600 nm at the reaction vessel.

In one embodiment, peak radiation in the 300-600 nm band is achieved by selecting a lamp or solar simulator whose spectral power distribution exhibits a maximum within the 300-600 nm band under operating conditions.

In one embodiment, peak radiation in the 300-600 nm band is achieved by applying an optical filter to an otherwise broadband source, where the optical filter attenuates wavelengths outside the 300-600 nm band while maintaining an irradiance at the reaction vessel of about 50 mW/cm² to about 200 mW/cm².

In one embodiment, the irradiation comprises a composite spectrum produced by combining multiple sources, including one or more ultraviolet/visible emitters that provide a spectral maximum within 300-600 nm and one or more visible/near-infrared emitters that provide additional heating.

In one embodiment, the irradiation source is configured such that the 300-600 nm band overlaps an extinction feature attributable to dielectric Mie resonance of the copper-containing core particle.

In one embodiment, the spectral power distribution at the vessel is verified by measuring spectral irradiance at the vessel position, and the peak wavelength and/or peak band are recorded for the reaction condition.

In one embodiment, the light source is positioned relative to the reaction vessel and operated at a power setting selected to provide an irradiance at the reaction vessel of about 50 mW/cm² to about 200 mW/cm², with a measured spectral maximum in the 300-600 nm band.

### EXAMPLES

The following examples are provided to illustrate certain embodiments and are not intended to limit the scope of the claims. Unless otherwise indicated, reagents were commercially obtained and used without further purification.

### Example 1—Preparation of Cu₂O—Pd Hybrid Nanocatalyst

Cu₂O spherical nanoparticles were synthesized via a microemulsion method at room temperature. In a representative procedure, a 250 mL round-bottom flask was loaded with 52.5 mL of n-heptane and 7.5 mL of Brij® L-4 surfactant and stirred at approximately 500 rpm. Thereafter, 5.4 mL of an aqueous 0.1 M Cu(NO₃)₂ solution was added, and 5.4 mL of a 1 M N₂H₄ solution was introduced dropwise as a reducing agent. The mixture was maintained under stirring for about 12 hours. The emulsion was disrupted by addition of acetone, and the precipitate was collected by centrifugation. The precipitate was washed once with acetone and twice with ethanol to remove residual surfactant and then redispersed in 3 mL ethanol by sonication.

Palladium was deposited on the Cu₂O particles by adding 21.3 mL of an aqueous 0.5 mM K₂PdCl₄ solution to the ethanol dispersion under vigorous stirring for about 3 minutes. The resulting Cu₂O—Pd hybrid nanocatalyst was recovered by centrifugation, washed twice with ethanol and once with dimethylformamide (DMF), and stored for subsequent catalytic testing.

### Example 2—Preparation of CuO—Pd Hybrid Nanocatalyst

CuO nanoparticles were provided as a commercial material. A representative mass (32 mg) was washed once with acetone and twice with ethanol and recovered by centrifugation. The washed CuO was redispersed in 3 mL ethanol via sonication. Palladium was deposited by adding 21.3 mL of an aqueous 0.5 mM K₂PdCl₄ solution under vigorous stirring for about 3 minutes. The resulting CuO—Pd hybrid nanocatalyst was recovered by centrifugation, washed twice with ethanol and once with DMF, and stored for use.

### Example 3—Photothermal Suzuki Coupling Under Broadband Irradiation

Suzuki coupling between iodobenzene and phenylboronic acid to produce biphenyl was conducted in a 25 mL three-neck round-bottom flask equipped with two reflux condensers and a stopper. Cold water was circulated through the condensers and a continuous nitrogen flow was maintained to provide an inert atmosphere. The catalyst (Cu₂O—Pd or CuO—Pd) was dispersed in a solvent mixture consisting of 80% DMF and 20% deionized water (total volume 15 mL) and introduced into the flask.

To initiate the reaction, potassium carbonate (K₂CO₃, 1.5 mmol), phenylboronic acid (1.08 mmol), and iodobenzene (0.9 mmol) were added. The reaction mixture was irradiated with a broadband ELH 120V-300W lamp positioned about 2.5 cm from the flask to simulate approximately one-sun intensity (about 107.5 mW/cm²) over a wavelength range of about 300-2000 nm. Samples were analyzed by GC-MS using calibration standards to determine conversion and yield. Under these conditions, complete iodobenzene conversion was achieved within about 10 minutes. Biphenyl yields at 10 minutes were approximately 96.3% with Cu₂O—Pd and approximately 99.1% with CuO—Pd. Continued irradiation provided 100% yield within about 1 hour without external heating.

### Example 4—Suzuki Coupling Under Light-Off, Ambient Conditions

The Suzuki coupling of Example 3 was repeated under light-off, ambient (room temperature) conditions without external heating. The same solvent mixture (80% DMF/20% water), reagent amounts, and catalyst loadings were used. Under these conditions, the reaction proceeded more slowly but provided substantial product formation. After about 8 hours, biphenyl yields were approximately 90.97% with Cu₂O—Pd and approximately 73.9% with CuO—Pd, demonstrating that the hybrid catalysts remain active even without irradiation or external heat input.

### Example 5—Temperature-Matched Thermal Control

To evaluate whether irradiation-enhanced performance was attributable to photothermal heating, the Suzuki coupling of Example 3 was repeated under light-off conditions with external heating. A hot plate and an aluminum heat transfer block were used to heat the reaction mixture uniformly. The reaction was initiated after the catalyst dispersion reached approximately 113° C., comparable to a maximum bulk temperature of approximately 116° C. observed in light-on experiments. Under these temperature-matched thermal conditions, Cu₂O—Pd achieved complete conversion within about 10 minutes, and CuO—Pd achieved complete conversion within about 8 minutes. These results support that the light-on enhancement was primarily a thermal (photothermal) effect.

### Example 6—Catalyst Characterization and Post-Reaction Stability

Fresh Cu₂O—Pd and CuO—Pd hybrid nanocatalysts were characterized by X-ray diffraction (XRD), UV-Vis extinction spectroscopy, scanning electron microscopy (SEM), transmission electron microscopy (TEM), and energy-dispersive spectroscopy (EDS) mapping. Representative measurements indicated quasi-spherical copper oxide cores decorated with palladium nanoclusters having an average size of about 3±1 nm. Due to ultra-low Pd loading, characteristic Pd peaks were not observed in XRD patterns, while Pd presence was confirmed by microscopy and elemental mapping.

After completion of the photothermal coupling reaction (light-on), the catalysts were recovered (e.g., by centrifugation), washed, and re-characterized by XRD and TEM. Comparison of fresh and used catalysts indicated no significant phase changes and no discernible changes in particle morphology, supporting structural stability under reaction conditions.

### Example 7—Substrate Scope for Biaryl Formation

The procedure of Example 3 is performed with alternative aryl halides and/or arylboronic acids to form substituted biaryl products. Representative aryl halides include bromobenzene, chlorobenzene, 4-bromotoluene, 4-bromoanisole, 4-bromobenzaldehyde, 4-bromobenzonitrile, 4-bromopyridine, and 1-bromonaphthalene. Representative organoboron reagents include phenylboronic acid, 4-methylphenylboronic acid, 4-methoxyphenylboronic acid, and 4-fluorophenylboronic acid. Reaction conditions (e.g., base selection, catalyst loading, irradiation intensity, and solvent composition) are adjusted to optimize conversion and yield for the selected substrates. For less reactive substrates (e.g., aryl chlorides), increased irradiation intensity and/or elevated bulk temperature is employed.

### Example 8—Catalyst Recovery and Reuse

After a coupling reaction, the hybrid nanocatalyst is separated from the reaction mixture by centrifugation and washed sequentially with ethanol and DMF. The recovered catalyst is then reused in a subsequent Suzuki coupling run under the conditions of Example 3. Conversion and yield are monitored over at least three cycles to evaluate recyclability. XRD and TEM are performed after the final cycle to assess structural integrity.

### Example 9—Other C—C Coupling Reactions Enabled by Photothermal Heating

A hybrid copper oxide-Pd nanocatalyst as described herein is employed for other Pd-mediated coupling reactions (e.g., Sonogashira, Negishi, Kumada, or Stille coupling) in which a Pd catalytic cycle benefits from elevated temperature. The copper oxide component harvests broadband irradiation and generates photothermal heating, while Pd provides catalytically active sites. Reaction partners, base/cocatalyst selections, and solvents are selected according to the coupling type, and irradiation conditions are adjusted to reach target temperatures.

### Example 10—Scale-Up Under Simulated Solar Irradiation

The Suzuki coupling of Example 3 is scaled to a larger reactor volume by proportionally increasing reagent and solvent quantities while maintaining catalyst concentration. Irradiation is provided by one or more broadband lamps or concentrated solar irradiation. Reactor geometry, stirring, and heat management are selected to maintain uniform temperature and mass transfer. Product is isolated by conventional workup and purification steps suitable for the target biaryl compound.

### Analytical and Calculation Notes

Conversion is calculated from the initial reactant concentration [R]ₒ and the reactant concentration at time t, [R]ₜ, as: Conversion (%)=([R]ₒ−[R]ₜ)/[R]ₒ×100. Percent yield is calculated from the product concentration at time t, [P]ₜ, using the theoretical yield defined by [R]ₒ, as: Percent yield (%)=[P]ₜ/[R]ₒ×100. Unless otherwise stated, reported conversion and yield values correspond to averages of three independent experiments, with variability represented by error bars.

TABLE 1
List of reactants

| Aryl Halide (Ar—X) | Boronic Acid (Ar′—(OH)₂) | Expected Product |
|---|---|---|
| Bromobenzene | Phenylboronic acid | Biphenyl |
| 4-Bromotoluene | Phenylboronic acid | 4-Methylbiphenyl |
| 4-Bromoanisole | Phenylboronic acid | 4-Methoxybiphenyl |
| 4-Bromobenzaldehyde | Phenylboronic acid | 4-Formylbiphenyl |
| 4-Bromobenzonitrile | Phenylboronic acid | 4-Cyanobiphenyl |
| Chlorobenzene | Phenylboronic acid | Biphenyl |
| Iodobenzene | Phenylboronic acid | Biphenyl |
| 4-Bromotoluene | 4-Methylphenylboronic acid | 4,4′-Dimethylbiphenyl |
| 4-Bromotoluene | 4-Methoxyphenylboronic acid | 4-Methyl-4′-methoxybiphenyl |
| 4-Bromotoluene | 4-Fluorophenylboronic acid | 4-Methyl-4′-fluorobiphenyl |
| 4-Bromopyridine | Phenylboronic acid | Phenylpyridine |
| 1-Bromonaphthalene | Phenylboronic acid | Phenylnaphthalene |

### Results

The following results summarize representative experiments and observations and are provided as examples rather than limitations.

Catalyst characterization. Cu₂O—Pd and CuO—Pd hybrid nanocatalysts are characterized by SEM, TEM, HRTEM, EDS mapping, XRD, and UV-Vis extinction spectroscopy. Representative measurements show quasi-spherical copper oxide particles decorated with Pd nanoclusters (~3±1 nm). UV-Vis extinction spectra show optical features in a 400-600 nm region consistent with dielectric Mie resonance behavior of the copper oxide core. XRD patterns confirm copper oxide phases (Cu₂O or CuO) and show no detectable Pd peaks due to low Pd loading.

Photothermal coupling under broadband irradiation. In a representative Suzuki coupling (iodobenzene+phenylboronic acid; K₂CO₃ base; DMF/water solvent), irradiation with a broadband ELH 120V-300W lamp positioned about 2.5 cm from the flask provides approximately one-sun intensity (~107.5 mW/cm²) over about 300-2000 nm. Under light-on conditions, complete conversion is achieved within about 10 minutes with biphenyl yields of approximately 96% (Cu₂O—Pd) and approximately 99% (CuO—Pd). The reaction mixture warms to approximately 116° C. during illumination.

Ambient, light-off operation. Under light-off conditions at ambient temperature without external heating, coupling proceeds more slowly but remains detectable. For example, after about 8 hours, representative experiments show iodobenzene conversions of approximately 80% (Cu₂O—Pd) and approximately 62% (CuO—Pd), with corresponding substantial biphenyl formation.

Thermal control experiments. Thermal control runs under light-off conditions with external heating to a temperature comparable to the maximum light-on temperature (e.g., about 113° C.) show rapid conversion (e.g., complete conversion in about 8-10 minutes). These results support a conclusion that enhancement under irradiation is attributable primarily to photothermal heating. In some comparisons, externally heated light-off runs proceed faster than light-on runs because the thermal runs begin at an elevated temperature, whereas light-on runs begin near ambient temperature and warm gradually.

Catalyst stability. Post-reaction characterization (e.g., XRD and TEM) show no significant phase change or morphology change of copper oxide cores after light-on photothermal runs, indicating stability and suitability for reuse.

### Methods

The following methods are illustrative and may be modified for scale, substrates, reactor configuration, and analytical method.

Nanocatalyst synthesis (illustrative). Cu₂O spherical nanoparticles are synthesized via a microemulsion method at room temperature. In one procedure, a 250 mL round-bottom flask is loaded with 52.5 mL n-heptane and 7.5 mL Brij® L-4 surfactant and stirred at about 500 rpm. An aqueous 0.1 M Cu(NO₃)₂ solution (5.4 mL) is added, followed by dropwise addition of 5.4 mL of a 1 M N₂H₄ solution. The mixture is stirred for about 12 hours. The emulsion is disrupted with acetone, and precipitated Cu₂O is collected by centrifugation and washed (e.g., once with acetone and twice with ethanol). The Cu₂O is redispersed in ethanol (e.g., 3 mL) and Pd is deposited by adding an aqueous K₂PdCl₄ solution (e.g., 21.3 mL of 0.5 mM) under vigorous stirring (e.g., ~3 minutes). The resulting Cu₂O—Pd is recovered by centrifugation and washed (e.g., twice with ethanol and once with DMF).

CuO—Pd is prepared by washing commercial CuO nanoparticles (e.g., acetone and ethanol), redispersing in ethanol, and depositing Pd using the same K₂PdCl₄ procedure.

Catalyst characterization (illustrative). XRD is collected using Cu Kα radiation over a 20 range of about 20°-100° with step size ~0.02°. UV-Vis extinction spectra is collected in quartz cuvettes over about 200-1100 nm. SEM and TEM is used to assess morphology and Pd distribution, including EDS mapping. Conversion and yield is quantified by GC-MS using calibration standards.

Photoreactor setup and reaction conditions. Suzuki coupling is conducted in a 25 mL three-neck round-bottom flask equipped with reflux condensers and maintained under nitrogen. A catalyst dispersion is prepared in a solvent mixture (e.g., 80% DMF and 20% deionized water; total 15 mL) and added to the flask. Potassium carbonate (e.g., 1.5 mmol), phenylboronic acid (e.g., 1.08 mmol), and iodobenzene (e.g., 0.9 mmol) are added to initiate the reaction. Reactions are evaluated under: (i) light-on broadband irradiation (e.g., 300-2000 nm) from an ELH 120V-300W lamp positioned ~2.5 cm from the flask to simulate approximately one-sun intensity (~107.5 mW/cm²); (ii) light-off at room temperature; and (iii) light-off with external heating to a target temperature (e.g., ~113° C.).

Calculations. Conversion (%) is calculated from initial reactant concentration [R]ₒ and reactant concentration at time t, [R]ₜ using: Conversion=([R]ₒ−[R]ₜ)/[R]ₒ×100. Percent yield (%) is calculated from product concentration [P]ₜ and theoretical yield defined by [R]ₒ using: Percent yield=[P]ₜ/[R]ₒ×100. In certain experiments, reported values correspond to averages of three independent experiments with variability represented by error bars.

What is claimed is:

1. A hybrid dielectric Mie resonator nanocatalyst comprising: (a) a copper-containing core particle comprising cuprous oxide (Cu₂O), cupric oxide (CuO), metallic copper (Cu⁰), or any mixture thereof; and (b) palladium disposed at or near a surface of the copper-containing core particle as nanoclusters, nanoparticles, islands, a discontinuous coating, a thin film, a partial thin film, or any combination thereof; wherein the copper-containing core particle exhibits a dielectric Mie resonance feature in a wavelength region of about 400 nm to about 600 nm; and wherein a palladium loading is less than about 4 wt % relative to a total mass of the hybrid dielectric Mie resonator nanocatalyst; wherein the hybrid dielectric Mie resonator nanocatalyst catalyzes a carbon-carbon cross-coupling reaction between an aryl electrophile and an organoboron reagent under broadband irradiation without external heating; and wherein the hybrid dielectric Mie resonator nanocatalyst catalyzes the carbon-carbon cross-coupling reaction without irradiation and without external heating with an extended reaction time.

2. The hybrid dielectric Mie resonator nanocatalyst of claim 1, wherein the copper-containing core particle comprises Cu₂O nanoparticles prepared by a microemulsion process.

3. The hybrid dielectric Mie resonator nanocatalyst of claim 1, wherein the copper-containing core particle comprises CuO.

4. The hybrid dielectric Mie resonator nanocatalyst of claim 1, wherein the copper-containing core particle comprises a mixture of Cu₂O and CuO.

5. The hybrid dielectric Mie resonator nanocatalyst of claim 1, wherein the copper-containing core particle comprises a mixture of Cu₂O, CuO, and metallic copper (Cu⁰).

6. The hybrid dielectric Mie resonator nanocatalyst of claim 1, wherein the palladium comprises nanoclusters having an average size of about 1 nm to about 10 nm.

7. The hybrid dielectric Mie resonator nanocatalyst of claim 1, wherein the palladium forms the thin film over the copper-containing core particle.

8. The hybrid dielectric Mie resonator nanocatalyst of claim 1, wherein the palladium forms the partial thin film over the copper-containing core particle.

9. The hybrid dielectric Mie resonator nanocatalyst of claim 1, wherein the copper-containing core particle has an average diameter of about 5 nm to about 500 nm and is quasi-spherical or spherical.

10. The hybrid dielectric Mie resonator nanocatalyst of claim 1, wherein the copper-containing core particle is cubical.

11. The hybrid dielectric Mie resonator nanocatalyst of claim 1, wherein palladium is deposited from an aqueous K₂PdCl₄ precursor onto the copper-containing core particle.

12. The hybrid dielectric Mie resonator nanocatalyst of claim 1, wherein the hybrid dielectric Mie resonator nanocatalyst is a heterogeneous solid separable from a reaction mixture by centrifugation or filtration.

13. A method of forming a biaryl compound, the method comprising: contacting an aryl electrophile and an arylboronic acid with the hybrid dielectric Mie resonator nanocatalyst of claim 1 in the presence of a base and a solvent system to form a reaction mixture; and maintaining the reaction mixture under conditions effective to form the biaryl compound, wherein maintaining comprises either (i) irradiating the reaction mixture with broadband irradiation to promote photothermal coupling while supplying no external heating to the reaction mixture, or (ii) conducting the reaction mixture without irradiation and without external heating.

14. The method of claim 13, wherein the irradiating comprises broadband irradiation comprising wavelengths of about 300 nm to about 2000 nm.

15. The method of claim 13, wherein an irradiance at a reaction vessel is about 50 mW/cm² to about 200 mW/cm² and the irradiating is provided by an irradiation source that exhibits peak radiation within about 300 nm to about 600 nm at the reaction vessel.

16. The method of claim 13, wherein the solvent system comprises N,N-dimethylformamide and water, or comprises ethanol and water; and wherein the base is selected from carbonates, hydroxides, phosphates, alkoxides, and amines.

17. The method of claim 13, wherein the aryl electrophile comprises iodobenzene and the arylboronic acid comprises phenylboronic acid such that the biaryl compound comprises biphenyl; wherein the reaction mixture reaches a bulk temperature of at least about 80° C. under the broadband irradiation; and wherein the method provides at least about 90% yield of biphenyl within about 10 minutes of the irradiating when the irradiance at the reaction vessel is about 100 mW/cm² and the solvent system comprises about 80 vol % N,N-dimethylformamide and about 20 vol % water and the base comprises potassium carbonate.

18. A method of making a hybrid nanocatalyst, the method comprising: providing cupric oxide (CuO) nanoparticles; dispersing the CuO nanoparticles in an alcohol to form a dispersion; contacting the dispersion with an aqueous palladium (II) precursor solution to deposit palladium at or near surfaces of the CuO nanoparticles; and recovering a heterogeneous solid to provide a CuO—Pd hybrid nanocatalyst.

19. The method of claim 18, wherein the palladium (II) precursor comprises K₂PdCl₄, and wherein providing the cupric oxide nanoparticles comprises washing the cupric oxide nanoparticles with acetone and ethanol prior to the dispersing.

20. The method of claim 18, further comprising partially reducing at least a portion of copper in the recovered heterogeneous solid to form metallic copper (Cu⁰) while retaining CuO and/or Cu₂O in the recovered heterogeneous solid.
